(19) United States
(12) Patent Application Publication
(10) Pub. No.: US 2011/0226864 A1
(43) Pub. Date: Sep. 22, 2011

(54) MOBILE DEVICE AND METHOD FOR EMITTING FRAGRANCE

(75) Inventors: Young Jin KIM, Gumi-si (KR); Moon Heui LEE, Gumi-si (KR)

(73) Assignee: SAMSUNG ELECTRONICS CO. LTD., Suwon-si (KR)

(21) Appl. No.: 13/051,305

(22) Filed: Mar. 18, 2011

(30) Foreign Application Priority Data
Mar. 19, 2010 (KR) 10-2010-0024866

(51) Int. Cl. A61L 9/04; B01J 19/00

(52) U.S. Cl. 239/6; 422/107

(57) ABSTRACT

A method for emitting a fragrance in a mobile device and a mobile device for supporting a fragrance emitting function are provided. The mobile device includes a user database, a fragrance emitting module, and a control unit. The user database includes additive information related to one or more user functions of the mobile device. The fragrance emitting module emits at least one of a plurality of fragrances selected depending on a type of additive information. The control unit, when an event occurs that corresponds to at least one of the one or more user functions, determines whether the event is a specific event correlated with the additive information. The control unit also controls the fragrance emitting module to emit the at least one of the plurality of fragrances in response to the occurrence of the specific event.

### PRIORITY

This application claims the benefit under 35 U.S.C. §119(a) of a Korean patent application filed on Mar. 19, 2010 in the Korean Intellectual Property Office and assigned Serial No. 10-2010-0024866, the entire disclosure of which is hereby incorporated by reference.

### BACKGROUND OF THE INVENTION

1. Field of the Invention

The present invention relates to a mobile device. More particularly, the present invention relates to a mobile device including a fragrance emitting module that emits a particular fragrance corresponding to specific additive information stored in a memory unit when a given event occurs.

2. Description of the Related Art

Normally a mobile device refers to a type of electronic device that includes mobility and portability among its primary features. With the remarkable development of related technologies, a great variety of mobile devices such as a mobile communication device and a Personal Digital Assistant (PDA) are increasing in popularity. A typical mobile communication device has a module suitable for the execution of a mobile communication function and thereby allows voice and data communications with other mobile communication devices through a base station. A typical PDA has a Central Processing Unit (CPU), a memory unit, an Operating System (OS), and various programs and modules operating based on the OS and thereby allows various functions to collect, store, write and search information.

Like the PDA described above, a conventional mobile device supports a mobile communication function or a specific user function based on various programs stored in the memory unit. However, such a function may be limited to a visible or audible stage. More sensible and emotional operations and uses of a function are therefore required for an advanced mobile device.

### SUMMARY OF THE INVENTION

An aspect of the present invention is to address at least the above-mentioned problems and/or disadvantages and to provide at least the advantages described below. Accordingly an aspect of the present invention is to provide a mobile device and related method in which a fragrance emitting module emits a particular fragrance corresponding to specific additive information stored in a memory unit when a given event occurs.

In accordance with an aspect of the present invention, a mobile device for supporting a fragrance emitting function is provided. The mobile device includes a user database including additive information related to one or more user functions of the mobile device, a fragrance emitting module for emitting at least one of a plurality of fragrances selected depending on a type of additive information, and a control unit for, when an event occurs that corresponds to at least one of the one or more user functions, determining whether the event is a specific event correlated with the additive information, and for controlling the fragrance emitting module to emit the at least one of the plurality of fragrances in response to the occurrence of the specific event.

In accordance with another aspect of the present invention, a method for emitting a fragrance in a mobile device is provided. The method includes recognizing the occurrence of an event that corresponds to at least one of one or more user functions of the mobile device when a fragrance emitting mode is activated, determining whether the event is a specific event correlated with additive information stored in a user database, and emitting at least one of a plurality of fragrances depending on a type of the additive information.

In accordance with another aspect of the present invention, a mobile device and related method can allow a user to associate particular fragrances with various situations and thereby to more sensibly and emotionally use the functions of the mobile device.

Other aspects, advantages, and salient features of the invention will become apparent to those skilled in the art from the following detailed description, which, taken in conjunction with the annexed drawings, discloses exemplary embodiments of the invention.

### BRIEF DESCRIPTION OF THE DRAWINGS

The above and other aspects, features, and advantages of certain exemplary embodiments of the present invention will be more apparent from the following description taken in conjunction with the accompanying drawings, in which:

FIG. 1 is a block diagram illustrating a configuration of a mobile device according to an exemplary embodiment of the present invention;

FIG. 2 is a block diagram illustrating a detailed configuration of a control unit shown in FIG. 1 according to an exemplary embodiment of the present invention;

FIG. 3 is a flow diagram illustrating a method for emitting a fragrance in a mobile device according to an exemplary embodiment of the present invention;

FIG. 4 is a flow diagram illustrating a fragrance emitting method based on a character string in a mobile device according to an exemplary embodiment of the present invention; and

FIG. 5 is a flow diagram illustrating a fragrance emitting method based on group information in a mobile device according to an exemplary embodiment of the present invention.

Throughout the drawings, it should be noted that like reference numbers are used to depict the same or similar elements, features, and structures.

### DETAILED DESCRIPTION OF THE INVENTION

The following description with reference to the accompanying drawings is provided to assist in a comprehensive understanding of exemplary embodiments of the invention as defined by the claims and their equivalents. It includes various specific details to assist in that understanding but these are to be regarded as merely exemplary. Accordingly, those of ordinary skill in the art will recognize that various changes and modifications of the embodiments described herein can be made without departing from the scope and spirit of the invention. In addition, descriptions of well-known functions and constructions may be omitted for clarity and conciseness.

The terms and words used in the following description and claims are not limited to the bibliographical meanings, but, are merely used by the inventor to enable a clear and consistent understanding of the invention. Accordingly, it should be apparent to those skilled in the art that the following description of exemplary embodiments of the present invention is provided for illustration purpose only and not for the purpose of limiting the invention as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

Among terms set forth herein, an event refers to the generation of a signal for performing a particular function selected among various user functions supported by a mobile device. For instance, the event may correspond to a signal that is generated in the mobile device or received from the outside in order to perform a particular function such as a call or communication function, a file-related function, a camera-related function, an information collecting function, an information determination function based on a specific algorithm, and the like.

Also, additive information refers to specific information in connection with user functions of the mobile device stored in a user database. The mobile device offers the additive information when a selected user function is activated. For instance, the additive information may be group information to which a sender's number of a received message belongs.

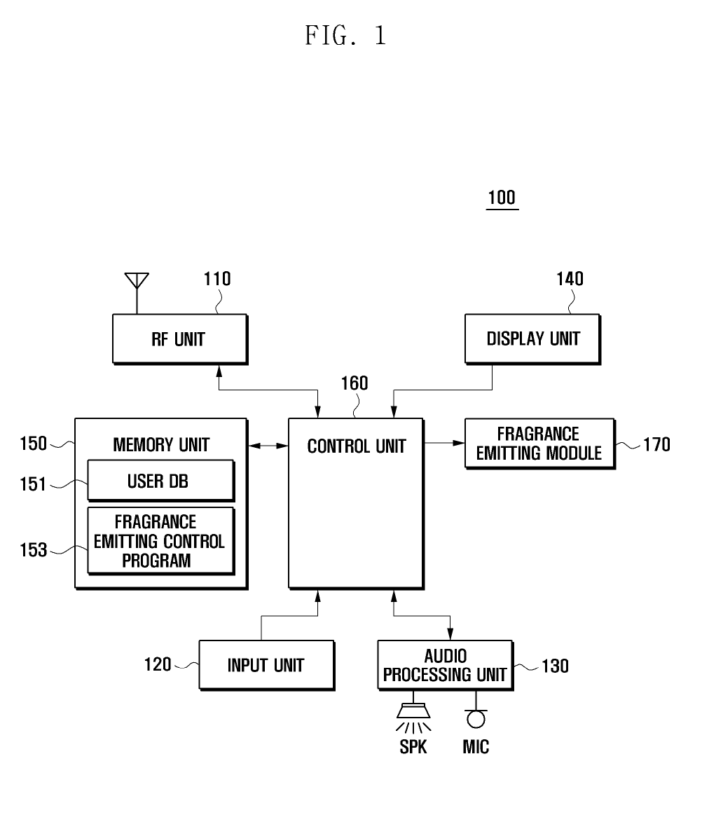

FIG. 1 is a block diagram illustrating a configuration of a mobile device according to an exemplary embodiment of the present invention.

Referring to FIG. 1, the mobile device 100 includes a Radio Frequency (RF) unit 110, an input unit 120, an audio processing unit 130, a display unit 140, a memory unit 150, a control unit 160, and a fragrance emitting module 170.

The mobile device 100 having the above elements emits a particular fragrance selected according to an event generated in the mobile device 100, based on both a user DataBase (DB) 151 for storing additive information and a fragrance emitting control program 153, which are both stored in the memory unit 150. Therefore, a user not only can recognize the emission of a particular fragrance as the activation of a specific user function, but also can use sensibly and emotionally such a specific user function. Now, each individual element of the mobile device 100 will be described in detail.

The RF unit 110 establishes communication channels necessary for a voice call, a video call, data transmission, etc. under the control of the control unit 160. Namely, the RF unit 110 forms a voice call channel, a video call channel, a data communication channel, etc. with a mobile communication system. Additionally, the RF unit 110 may include an RF transmitter that upwardly converts the frequency of signals to be transmitted and amplifies the signals, and an RF receiver that amplifies received signals with low-noise and downwardly converts the frequency of the received signals. For the operation of the RF unit 110, the mobile device 100 may control the display of various data or information such as phonebook information on the display unit 140. If the mobile device 100 does not support a mobile communication function, the RF unit 110 may be omitted from the mobile device 100. A message or call received by the RF unit 110 may be correlated with specific additive information in the user database 151 stored in the memory unit 150 and be associated to a specific group. Also, a message received by the RF unit 110 may be correlated with specific additive information in the user database 151 and recognized as a message containing a specific character string. For the above, the user database 151 may store additive information having given character string information and phonebook information.

The input unit 120 includes a plurality of input keys and function keys to receive a user's input and to set various functions. The function keys may have navigation keys, side keys, shortcut keys, and any other special keys defined to perform particular functions. If the mobile device 100 uses a full screen, the input unit 120 may have only side keys formed on sides of a device body without any key buttons or keypad. If the mobile device 100 uses a touch screen, the input unit 120 may be formed of a key map having a number of key icons to be arranged on the display unit 140 and also a touch panel setting based on the key map. The key map may include an alphanumeric key map, a QWERTY key map, a 3*4 key map, a 4*3 key map, a menu map, a soft key map, and the like. In particular, when a user inputs phonebook information and character string information contained in the additive information of the user database 151, the input unit 120 creates an input signal corresponding to a user's input and sends it to the control unit 160 to input the additive information. Additionally, the input unit 120 may have a special key that selects the activation or inactivation of a fragrance emitting mode for the operation of the fragrance emitting module 170. This special key may be assigned as a hot key or offered in the form of a menu item or key icon contained in a menu map by a user or device designer.

The audio processing unit 130 includes a speaker (SPK) for outputting various audio data received during a call, contained in a received message, or produced by a playback of an audio file stored in the memory unit 150, and a microphone (MIC) for collecting a user's voice and any other audio signals during a call. More particularly, when a specific event for activating the fragrance emitting module 170 occurs, the audio processing unit 130 may output predefined audio data for informing a user of the occurrence of an event. Also, the audio processing unit 130 may offer voice guidance for types and properties of a specific fragrance emitted when the event occurs. Such audio data or voice guidance may be omitted according to a user's choice.

The display unit 140 represents a variety of information inputted by a user or offered to a user, including various menus of the mobile device 100. For instance, the display unit 140 may visually offer various screen views of an idle screen, a menu screen, a message writing screen, a call screen, a game screen, a music playback screen, and the like. Additionally, the display unit 140 may utilize a Liquid Crystal Display (LCD), an Organic Light Emitting Diode (OLED), or any other equivalent. If an LCD or an OLED is fabricated in the form of a touch screen, the display unit 140 may act as an input unit as well.

More particularly, when a specific event occurs and if the event meets given conditions for activating a fragrance emitting function, the display unit 140 may offer information about the fragrance being emitted. This information may include information indicating that the event meets the conditions for emitting a specific fragrance, and information about types and properties of a specific fragrance being emitted. Also, the display unit 140 may offer a specific icon that allows a user to distinguish an activated or inactivated state of the fragrance emitting mode. Additionally, when the RF unit 110 receives a message, the display unit 140 may display a sender's number contained in the received message. Also, when the received message includes a specific character or character string contained in the additive information, the display unit 140 may display the specific character or character string to be distinguished from the others. And also, the display unit 140 may offer information about types and properties of a specific fragrance being emitted depending on the specific character or character string. Furthermore, when a message or call is received and if a sender's number belongs to a specific group of a phonebook as additive information, the display unit 140 may offer information indicating that the event meets conditions for emitting a specific fragrance. Here, the display unit 140 may also offer information about a specific fragrance corresponding to a specific group. As discussed above, when a current event in the mobile device 100 meets specific conditions for emitting a fragrance through a comparison with additive information of the user database 151 stored in the memory unit 150, the display unit 140 may offer corresponding audio data and information about types and properties of a fragrance being emitted.

The memory unit 150 stores a variety of applications required for functions related to exemplary embodiments of the present invention. If the mobile device 100 employs a touch screen, the memory unit 150 may store a key map, a menu map, etc. required for the operation of the touch screen. Here, the key map may have various well known types such as a keyboard map, a 3*4 key map, a QWERTY key map, or a special control key map suitable for the operation of a specific application in use. More particularly, the memory unit 150 may store the user database 151 and the fragrance emitting control program 153. The memory unit 150 may consist of a program region and a data region.

The program region may store an Operating System (OS) for booting and operating the mobile device 100, and applications required for performing various functions of the mobile device 100, such as a call application, a web browser for surfing on the Internet, a Moving Picture Experts Group (MPEG)-1 or MPEG-2 Audio Layer 3 (MP3) application for playing digital sounds, an image viewer application for showing image files, a video player application, and the like. More particularly, the program region stores the fragrance emitting control program 153.

The fragrance emitting control program 153 is loaded on the control unit 160 when a fragrance emitting mode is selected, and then controls the fragrance emitting module 170 so that a specific fragrance can be emitted when a particular event occurs. For this, the fragrance emitting control program 153 may contain a routine for identifying a type of current event, a routine for comparing a current event with additive information stored in the user database 151, a routine for determining whether a current event meets given conditions for emitting a fragrance, and a routine for controlling the fragrance emitting module 170 to emit a specific fragrance in response to a current event. Here, the control routine for the fragrance emitting module 170 may include a sub-routine for selecting the type of a fragrance and a sub-routine for selecting the strength of a fragrance.

The data region stores data and contents created or received while the mobile device 100 is used. In addition, if the display unit 140 is formed of a touch screen, the data region may store user's inputs received from the touch screen. More particularly, when any event for the operation of a user function occurs in the mobile device 100, the data region acts as a buffer that temporarily stores the event. Meanwhile, the user database 151 may store additive information including phonebook information and character string information. Also, the user database 151 may further store various algorithms and basic information for driving a selected algorithm as additive information. For instance, the algorithm may be an algorithm for checking a biorhythm, and basic information may be the date of birth required for a biorhythm check. Also, the algorithm may be a face recognition algorithm, and the basic information may be feature points of a user's face or other faces designated by a user. For supporting such a face recognition function, the mobile device 100 may further include a camera module.

The fragrance emitting module 170 is configured to emit a particular fragrance under the control of the control unit 160. The fragrance emitting module 170 may include a storage space in which a source substance for producing a fragrance is kept, and a spraying outlet through which the source substance is emitted. The storage space may have a valve that allows a uniform spraying of the source substance through the spraying outlet, and a pressing plate that applies a uniform pressure to the source substance. The fragrance emitting module 170 may have several storage spaces that keep different source substances, and a mixing space in which such source substances are selectively mixed with each other to produce various fragrances with various strengths. Alternatively, the fragrance emitting module 170 may selectively spray the source substances through the spraying outlet under the control of the control unit 160. Meanwhile, the fragrance emitting module 170 may have a sensor for sensing the remaining amount of each substance. When a certain substance is reduced below a given level, the control unit 160 may receive related information from the sensor and then turn on an alarm for a recharge of the reduced substance.

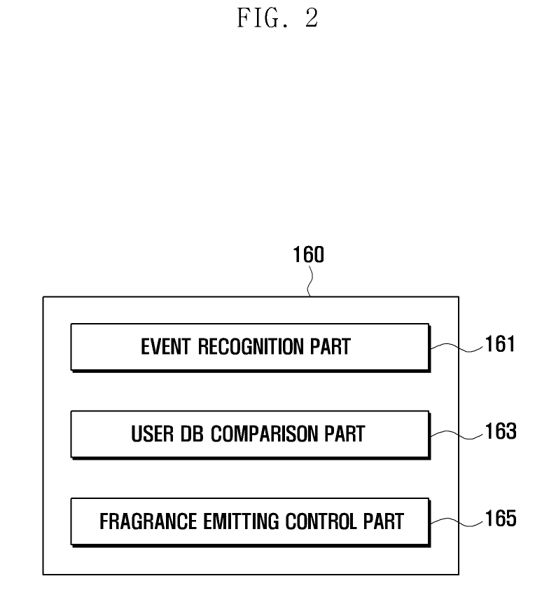

The control unit 160 controls a power supply and initializing process for each element of the mobile device 100. After initializing, the control unit 160 may support the fragrance emitting function for each element of the mobile device 100. For this, the control unit 160 may include the elements shown in FIG. 2.

FIG. 2 is a block diagram illustrating a detailed configuration of a control unit shown in FIG. 1 according to an exemplary embodiment of the present invention.

Referring to FIG. 2, the control unit 160 may include an event recognition part 161, a user database comparison part 163, and a fragrance emitting control part 165.

The event recognition part 161 recognizes various events that can occur, such as a message reception event, a call reception event, a face information acquisition event, a biorhythm information acquisition event, and the like. The message reception event includes an event to receive a message containing a sender's number, a message containing a character string, or a message containing a code for instructing a fragrance to be emitted. The face information acquisition event includes an event to detect feature points of a face by performing face recognition for a face image captured through a camera module.

When the event recognition part 161 recognizes a specific event, the user database comparison part 163 compares the recognized event with the additive information in the user database 151 and then sends comparison results to the fragrance emitting control part 165. More specifically, when the recognized event is an event to receive a message containing a sender's number, the user database comparison part 163 compares the sender's number with phonebook information stored in the user database 151 and then extracts information about a specific group to which the sender's number belongs. Additionally, when the recognized event is an event to receive a message containing a character string, the user database comparison part 163 compares the character string with a specific character string stored in the user database 151 and then extracts information about the specific character string. In this case, if two or more strings corresponding to the specific string stored in the user database 151 are detected, the user database comparison part 163 further detects information about frequency of such strings and sends it to the fragrance emitting control part 165. Also, when the recognized event is an event to receive a message or data containing a code for instructing a fragrance to be emitted, the user database comparison part 163 sends the code to the fragrance emitting control part 165. Also, when feature points are detected from a face image captured through a camera module, the user database comparison part 163 compares the detected feature points with pre-stored feature points and then sends coincident information to the fragrance emitting control part 165. And also, when a biorhythm information acquisition event occurs, the user database comparison part 163 compares the biorhythm information with pre-stored information and then sends comparison results to the fragrance emitting control part 165.

The fragrance emitting control part 165 receives comparison results from the user database comparison part 163 and, based on the received results, determines which kind of fragrance will be emitted. At this time, the fragrance emitting control part 165 may create a code for emitting a specific kind of fragrance and then send the created code to the fragrance emitting module 170. Also, the fragrance emitting control part 165 may add information about mixing sources and ratios of a specific fragrance to the created code. For this, the memory unit 150 may store information about mixing sources and ratios of a specific fragrance and then offer it to the fragrance emitting control part 165. Meanwhile, the fragrance emitting control part 165 may output information about the remaining amount of source substances kept in the fragrance emitting module to the display unit 140 and/or the audio processing unit 130 when the remaining amount reaches the critical level and/or when an input signal for checking the remaining amount is created.

As discussed heretofore, the mobile device 100 according to an exemplary embodiment of the present invention compares a current event with the additive information stored in the user database 151 and then controls the fragrance emitting module 170 to emit a particular fragrance according to comparison results. Namely, the mobile device 100 allows a user to feel a specific fragrance correlated with the occurrence of a particular event. Therefore, a user can easily and highly recognize the occurrence of a specific event and also sensibly and emotionally react to that.

Now, a method for emitting a particular fragrance in a mobile device will be described in detail.

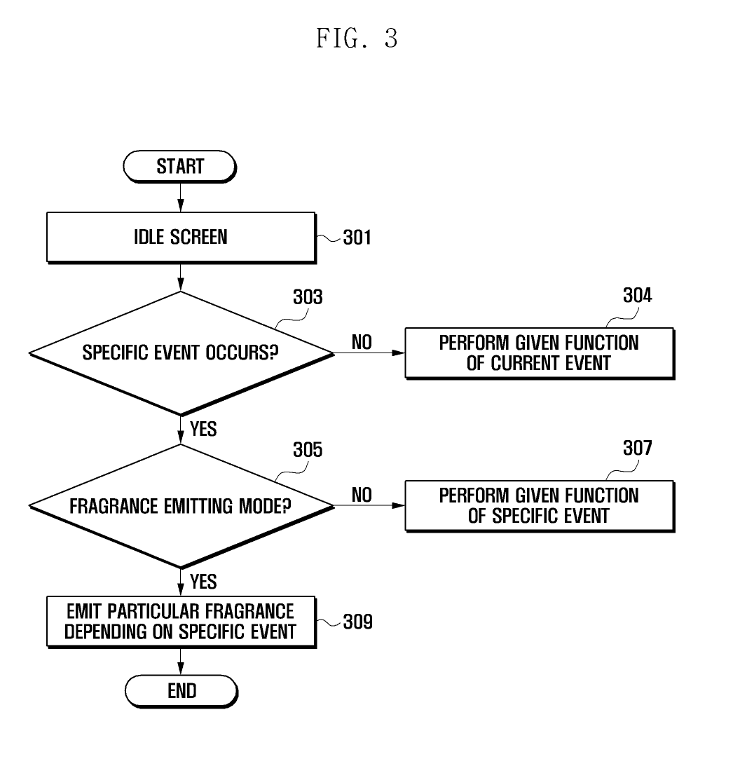

FIG. 3 is a flow diagram illustrating a method for emitting a fragrance in a mobile device according to an exemplary embodiment of the present invention.

Referring to FIG. 3, when power is supplied, the control unit 160 of the mobile device 100 performs a booting process and initializes its respective elements. Then the control unit 160 outputs a predefined idle screen on the display unit 140 in step 301.

Next, when a certain event occurs in the mobile device 100, the control unit 160 determines whether a current event is a specific event correlated with additive information in the user database 151 in step 303. If the current event is not correlated with the additive information, the control unit 160 performs a given function corresponding to the current event, such as a normal call function, a web access function, a file download function, a menu search function, and the like in step 304.

If the current event is a specific event correlated with the additive information in the user database 151, the control unit 160 further determines whether a fragrance emitting mode should be activated in step 305. Here, the specific event is an event correlated with the additive information stored in the user database 151. Additionally, the additive information may include group information in a phonebook used to determine whether a sender's number contained in a received message or call belongs to a specific group, character string information used to determine whether a received message contains a specific character string, code information used to determine whether a received message contains a specific code, feature point information used to identify feature points in a face image, biorhythm information used to compare with acquired biorhythm information, and the like. If the fragrance emitting mode should not be activated, the control unit 160 performs a given function corresponding to the current event, such as the display of a received message and group information, an alarm associated with the occurrence of an event, and the like in step 307.

If the fragrance emitting mode should be activated, the control unit 160 controls the emission of a particular fragrance in response to a specific event in step 309. In this step, the control unit 160 may control the emission of various kinds of fragrances according to events and also, even in the same event, control the emission of different kinds of fragrances depending on the circumstances. For instance, when receiving a message, the control unit 160 may control the emission of different fragrances according to a group to which a sender's number belongs. Also, when an event related to a character string occurs, the control unit 160 may control the emission of different fragrances according to a detected string. Related descriptions will be given later.

While the determination of whether the fragrance emitting mode should be activated is performed after the determination that an event correlated with the additive information occurs in the above description of the flow diagram of FIG. 3, the present invention is not limited thereto. Alternatively, the determination of whether the fragrance emitting mode should be activated may precede the determination of whether the specific event correlated with additive information occurs. In addition, if the fragrance emitting mode should not be activated, the aforesaid steps 307 and 304 may be selectively performed depending on the type of a current event.

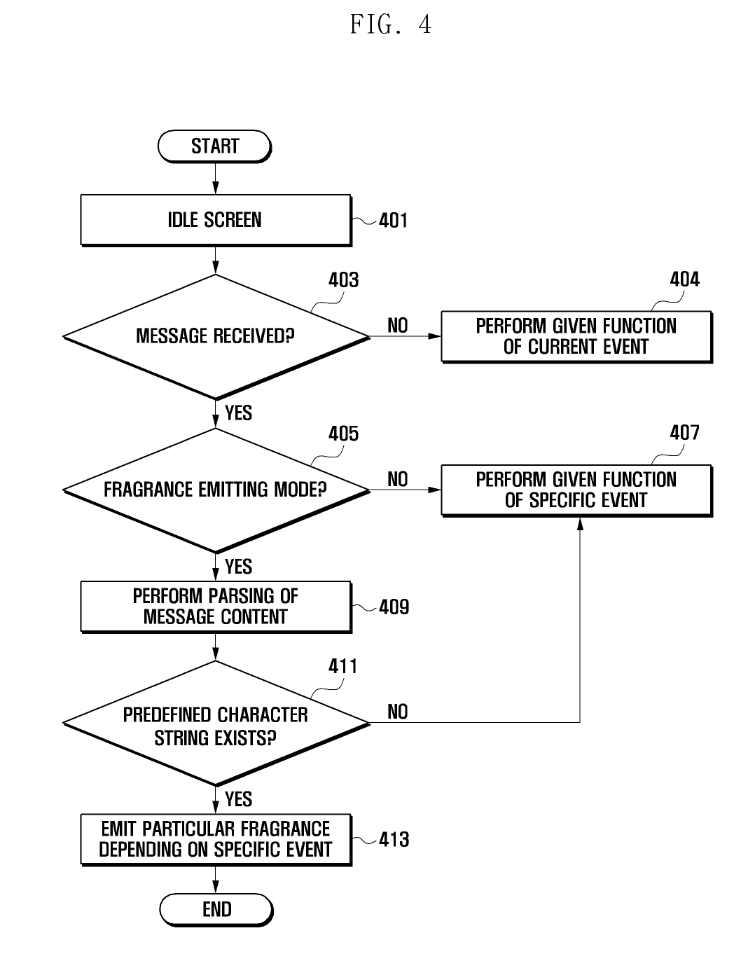

FIG. 4 is a flow diagram illustrating a fragrance emitting method based on a character string in a mobile device according to an exemplary embodiment of the present invention.

Referring to FIG. 4, when power is supplied, the control unit 160 of the mobile device 100 performs a booting process and initializes its respective elements. Then the control unit 160 outputs a predefined idle screen on the display unit 140 in step 401.

Next, when a certain event for a user function occurs in the mobile device 100, the control unit 160 determines whether a current event is a message reception event in step 403. If the current event is not the message reception event, the control unit 160 performs a given function corresponding to the current event, such as a normal call function, a web access function, a file download function, and the like in step 404.

If the current event is the message reception event, the control unit 160 further determines whether a fragrance emitting mode should be activated in step 405. If the fragrance emitting mode should not be activated, the control unit 160 performs a given function corresponding to the current event, such as outputting a received message in step 407.

If the fragrance emitting mode should be activated, the control unit 160 performs a parsing of the content of the received message in step 409. In this step, the control unit 160 may find information about a predefined character string contained in the user database 151 and also determine whether the received message contains any character string information. Then the control unit 160 determines whether the predefined character string exists in the received message in step 411. If there is no predefined character string in the received message, the control unit 160 may perform the aforesaid step 407.

If the predefined character string exists in the received message, the control unit 160 controls the emission of a particular fragrance depending on the predefined character string in step 413. In this step, if two or more different strings are detected together, the control unit 160 may control emission of a particular fragrance assigned to a character string with a higher weight factor (e.g., a higher frequency) or a higher priority. Additionally, if two or more of the same or different strings are detected repeatedly, the control unit 160 may control continuous emission of the corresponding same or different fragrances at regular intervals according to the order of strings contained in the message.

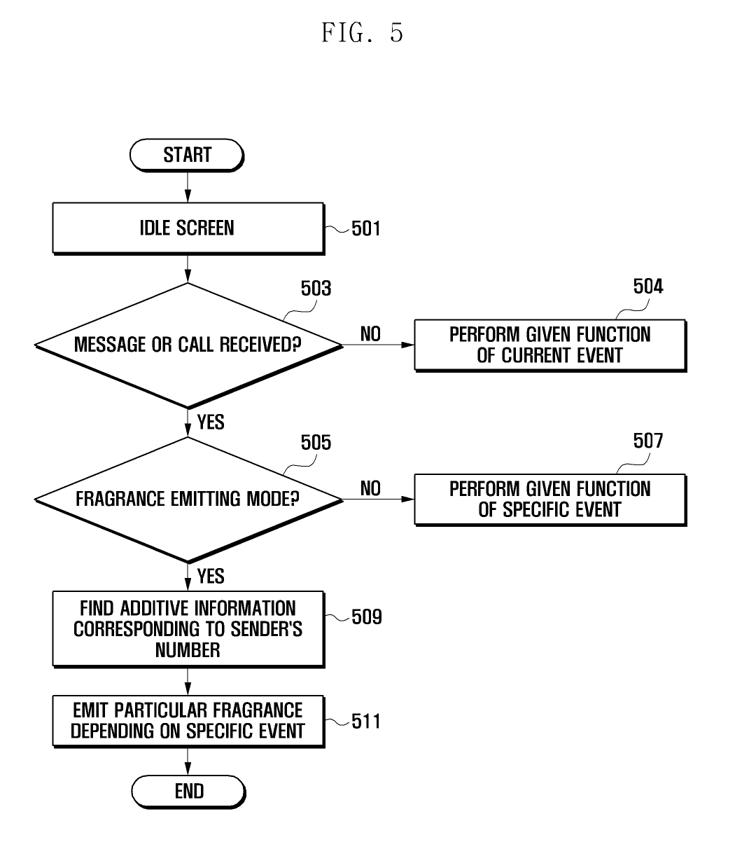

FIG. 5 is a flow diagram illustrating a fragrance emitting method based on group information in a mobile device according to an exemplary embodiment of the present invention.

Referring to FIG. 5, when power is supplied, the control unit 160 of the mobile device 100 performs a booting process and initializes its respective elements. Then the control unit 160 outputs a predefined idle screen on the display unit 140 in step 501.

Next, when a certain event for a user function occurs in the mobile device 100, the control unit 160 determines whether a current event is a message or call reception event in step 503. If the current event is not the message or call reception event, the control unit 160 performs a given function corresponding to the current event, such as a web access function, a widget information determination function, and the like in step 504.

If the current event is the message or call reception event, the control unit 160 further determines whether a fragrance emitting mode should be activated in step 505. If the fragrance emitting mode should not be activated, the control unit 160 performs a given function corresponding to the current event, such as outputting a received message or establishing a call in step 507.

If the fragrance emitting mode is activated, the control unit 160 finds additive information (e.g., group information) to which a sender's number contained in the received message or call belongs in step 509. In this step, the additive information may include group information to which the sender's number belongs, and phonebook information (such as nickname information, birthday information, intimacy information, etc.) related to the sender's number. The intimacy information may be set by a user or varied according to a call frequency.

Next, the control unit 160 controls emission of a particular fragrance depending on the additive information in step 511. For instance, if a phonebook has several groups of phone numbers and if a sender's number belongs to a specific group, the control unit 160 may emit a particular fragrance assigned to the specific group. Therefore, a user can infer a specific group of the received message or call from the particular fragrance emitted.

As discussed heretofore, the fragrance emission method of the mobile device determines, based on the pre-established user database 151, whether a currently occurring event meets predefined specific conditions, and then controls emission of a particular fragrance according to the conditions.

In the mobile device and method of exemplary embodiments of the present invention, the type of a particular fragrance emitted depending on a specific event may be varied according to a user's choice. For this, the mobile device 100 may offer a suitable menu page for selecting the types of fragrances corresponding to respective events. Therefore, for example, a user can designate particular fragrances for respective groups registered in the phonebook. Also, the type of a particular fragrance for each event may be set by a designer of the mobile device. In this case, the control unit 160 may control emission of a particular fragrance depending on the type of a specific event, for example, depending on feature points extracted through face recognition. Namely, the control unit 160 identifies an angry face, a happy face, a sad face, etc. by using feature points thereof and then controls emitting of a particular fragrance corresponding to each face.

Meanwhile, while the fragrance emitting function is performed in the above-discussed FIGS. 3 to 5 when an event occurs at the idle screen, the present invention is not limited thereto. Alternatively, the fragrance emitting function may be performed even while a user uses any other function of the mobile device 100. For instance, when a message reception event occurs during a music or video play, the mobile device 100 may execute the fragrance emitting function discussed in FIGS. 3 to 5. Also, the step of determination of whether the fragrance emitting mode should be activated may be performed before or after the step of determining the type of a current event.

In connection with a feature point detection event, a camera module may be enabled when the fragrance emitting mode is activated. Also, a typical face recognition function is enabled. Then the mobile device 100 extracts feature points from a face recognized image and thereby identifies the type of a face through the extracted feature points. For instance, the mobile device 100 may identify an angry face, a happy face, a sad face, etc. by using the feature points. For this, the user database 151 of the memory unit 150 may store information about feature points of various faces as additive information. When the type of face is identified, the mobile device 100 controls the fragrance emitting module 170 to emit a particular fragrance assigned to the identified face type.

In connection with a biorhythm detection event, a function to check biorhythm may be enabled and then related basic information such as birthday information may be inputted. Here, the mobile device 100 stores a suitable algorithm for checking biorhythm in the user database 151. This algorithm may be enabled by a user's request. Thereafter, when a given value of biorhythm is detected, for example, when a physical cycle reaches the maximum value or when an emotional cycle reaches the minimum value, the mobile device 100 controls the fragrance emitting module 170 to emit a particular fragrance assigned to the detected value of biorhythm.

In connection with a code detection event, the mobile device 100 may store a code table as the additive information in the user database 151 of the memory unit 150. The code table contains a number of codes each of which is an instruction for a particular fragrance to be emitted. Preferably, these codes may be prearranged between the mobile devices having the fragrance emitting module 170 or between the mobile device and a relevant server. If the mobile device 100 receives a message or data containing a code from the outside, the mobile device 100 checks the user database 151 to determine whether the code is valid. In a case of a valid code, the mobile device 100 identifies a particular fragrance corresponding to the code and then controls the fragrance emitting module 170 to emit a particular fragrance assigned to the code. Meanwhile, in order to prevent indiscreet or malicious emission of fragrances, the mobile device 100 may display a pop-up window that inquires of a user whether to perform the fragrance emitting function. The display of this pop-up window may have higher priority than a mode selection so that it can be executed even though the fragrance emitting mode is enabled.

The above-discussed mobile device 100 may essentially or selectively include any other elements. For instance, the mobile device 100 may further include a short range communication module, a digital camera module, a wired or wireless data transmission interface, an Internet access module, a digital broadcast receiving module, and so forth. According to trends in digital convergence, such elements may be varied, modified and improved in various ways, and any other elements equivalent to the above elements may be additionally or alternatively equipped in the mobile device 100. Meanwhile, as will be understood by those skilled in the art, some of the above-mentioned elements in the mobile device 100 may be omitted or replaced with another.

Additionally, the mobile device 100 of exemplary embodiments of the present invention may include any type of electronic device that has the fragrance emitting module 170 and the user database 151. For instance, the mobile device 100 may include communication devices, multimedia players and their application equipment, especially including many mobile communication terminals based on various communication protocols, a Portable Multimedia Player (PMP), a digital broadcasting player, a Personal Digital Assistant (PDA), a music player (e.g., an MP3 player), a portable game console, a smart phone, a notebook, a handheld personal computer, etc.

While this invention has been shown and described with reference to an exemplary embodiment thereof, it will be understood by those skilled in the art that various changes in form and details may be made therein without departing from the spirit and scope of the invention as defined by the appended claims and their equivalents.

What is claimed is:

1. A mobile device for supporting a fragrance emitting function, the mobile device comprising:
a user database including additive information related to one or more user functions of the mobile device;
a fragrance emitting module for emitting at least one of a plurality of fragrances selected depending on a type of additive information; and
a control unit for, when an event occurs that corresponds to at least one of the one or more user functions, determining whether the event is a specific event correlated with the additive information, and for controlling the fragrance emitting module to emit the at least one of the plurality of fragrances in response to the occurrence of the specific event.

2. The mobile device of claim 1, wherein the fragrance emitting module comprises:
a storage space in which source substances for producing each of the plurality of fragrances are kept; and
a spraying outlet through which at least one of the source substances are emitted.

3. The mobile device of claim 2, wherein the control unit selects the at least one of the source substances and adjusts the strength of the selected at least one of the source substances in order to emit the at least one of the plurality of fragrances.

4. The mobile device of claim 1, wherein the control unit comprises:
an event recognition part for recognizing the event;
a user database comparison part for comparing information associated with the recognized event with the additive information stored in the user database; and
a fragrance emitting control part for receiving comparison results from the user database comparison part and for controlling the fragrance emitting module to emit the at least one of the plurality of fragrances according to the received comparison results.

5. The mobile device of claim 4, wherein, when the event comprises receiving a message, the user database comparison part detects at least one character string corresponding to character string information contained in the additive information, and
wherein the fragrance emitting control part controls, when a single type of character string is detected, to emit the at least one of the plurality of fragrances corresponding to the detected character string, and controls, when two or more types of character strings are detected, to emit the at least one of the plurality of fragrances that correspond to the detected character strings according to at least one of a priority, a frequency and an order of the character strings.

6. The mobile device of claim 4, wherein, when the event comprises receiving one of a message and a call, the user database comparison part detects a sender's number contained in the received one of the message and the call and detects group information corresponding to the sender's number in the additive information, and
wherein the fragrance emitting control part controls the emitting of the at least one of the plurality of fragrances that correspond to the group information.

7. The mobile device of claim 4, wherein, when the event comprises receiving one of a message and a data containing a code for instructing a fragrance to be emitted, the user database comparison part detect a specific code corresponding to the received code in the additive information, and
wherein the fragrance emitting control part controls the emitting of the at least one of the plurality of fragrances that corresponds to the specific code.

8. The mobile device of claim 7, further comprising at least one of:
a display unit for displaying a pop-up window inquiring whether to perform the fragrance emitting function; and
an audio processing unit for outputting audio data inquiring whether to perform the fragrance emitting function.

9. The mobile device of claim 4, wherein, when the event comprises acquiring a face image, the user database comparison part detects feature points in the face image and compares the detected points with specific feature points contained in the additive information, and
wherein the fragrance emitting control part further controls the emitting of the at least one of the plurality of fragrances that corresponds to the specific feature points according to the comparison results.

10. The mobile device of claim 4, wherein, when the event comprises acquiring biorhythm information, the user database comparison part compares the acquired biorhythm information with specific biorhythm information contained in the additive information, and
wherein the fragrance emitting control part further controls the emitting of the at least one of the plurality of fragrances that corresponds to the specific biorhythm information according to the comparison results.

11. The mobile device of claim 1, further comprising:
a display unit for displaying information about a remaining amount of source substances that are used for emitting each of the plurality of fragrances.

12. A method for emitting a fragrance in a mobile device, the method comprising:
recognizing the occurrence of an event that corresponds to at least one of one or more user functions of the mobile device when a fragrance emitting mode is activated;
determining whether the event is a specific event correlated with additive information stored in a user database; and
emitting at least one of a plurality of fragrances depending on a type of the additive information.

13. The method of claim 12, wherein the emitting of the at least one of the plurality of fragrances comprises:
when the event comprises receiving a message, comparing character strings contained in the received message with character string information contained in the additive information; and
when at least one character string corresponding to the character string information contained in the additive information is detected, emitting the at least one of the plurality of fragrances in response to the detected character string.

14. The method of claim 13, wherein the emitting of the at least one of the plurality of fragrances comprises one of:
when a single type of character string is detected, emitting the at least one of the plurality of fragrances corresponding to the detected character string; and
when two or more types of character strings are detected, emitting the at least one of the plurality of fragrances corresponding to the detected character strings according to at least one of a priority, a frequency and an order of the character strings.

15. The method of claim 12, wherein the emitting of the at least one of the plurality of fragrances comprises:
when the event comprises receiving one of a message and a call, detecting a sender's number contained in the received message or call, detecting group information corresponding to the sender's number in the additive information, and emitting the at least one of the plurality of fragrances corresponding to the group information.

16. The method of claim 12, wherein the emitting of the at least one of the plurality of fragrances comprises:
when the event comprises receiving one of a message and data containing a code for instructing a fragrance to be emitted, detecting a specific code corresponding to the received code in the additive information, and emitting the at least one of the plurality of fragrances corresponding to the specific code.

17. The method of claim 12, wherein the emitting of the at least one of the plurality of fragrances comprises:
when the event comprises acquiring a face image, detecting feature points in the face image, comparing the detected points with specific feature points contained in the additive information, and emitting the at least one of the plurality of fragrances corresponding to the specific feature points according to the comparison results.

18. The method of claim 12, wherein the emitting of the at least one of the plurality of fragrances comprises:
when the event comprises acquiring biorhythm information, comparing the acquired biorhythm information with specific biorhythm information contained in the additive information, and emitting the at least one of the plurality of fragrances corresponding to the specific biorhythm information according to the comparison results.

19. The method of claim 12, further comprising at least one of:
outputting at least one of a pop-up window and audio data inquiring whether to perform the fragrance emitting function; and
outputting information about a remaining amount of source substances that are used for emitting each of the plurality of fragrances.
